(19) United States
(12) Patent Application Publication
(10) Pub. No.: US 2015/0087869 A1
(43) Pub. Date: Mar. 26, 2015

(54) CAPTURE OF TRIFLUOROMETHANE USING MOLECUAR SIEVES

(71) Applicant: E I DU PONT DE NEMOURS AND COMPANY, Wilmington, DE (US)

(72) Inventors: David Richard Corbin, West Chester, PA (US); Mark Brandon Shiflett, Wilmington, DE (US)

(73) Assignee: E I DU PONT DE NEMOURS AND COMPANY, Wilmington, DE (US)

(21) Appl. No.: 14/032,208

(22) Filed: Sep. 20, 2013

(51) Int. Cl. B01D 53/02; C07C 17/389

(52) U.S. Cl. CPC B01D 53/02; C07C 17/389; B01D 2253/102; B01D 2257/2066
USPC 570/179

(57) ABSTRACT

A method for capturing trifluoromethane from a gaseous mixture in a vent stream from a chlorodifluoromethane manufacturing process is described. In the method, the gaseous mixture is contacted with a molecular sieve, such as a zeolite or activated carbon, having a pore opening of at least about 5 Angstroms and a Sanderson electronegativity of less than or equal to about 2.75. The method is useful for reducing emissions of trifluoromethane, which has a high global warming potential.

This application claims priority under 35 U.S.C. §119(e) from, and claims the benefit of, U.S. Provisional Application No. 61/708,651 filed 2 Oct. 2012, which is by this reference incorporated in its entirety as a part hereof for all purposes.

### TECHNICAL FIELD

The invention relates to the field of greenhouse gas emission reduction. More specifically, the invention provides a method for capturing trifluoromethane from a gaseous mixture using molecular sieves, such as zeolites or activated carbon.

### BACKGROUND

Chlorodifluoromethane (R-22) is widely used as a propellant and refrigerant, and is also a versatile intermediate in the synthesis of organofluorine compounds. Chlorodifluoromethane is typically prepared by reacting chloroform with HF. A by-product of this reaction is trifluoromethane (R-23), which has a very high global warming potential (i.e., GWP=11,700 relative to CO₂ GWP=1). Therefore, methods to capture the trifluoromethane produced in the chlorodifluoromethane manufacturing process are needed to prevent its release into the atmosphere.

Zeolites are high capacity, selective sorbents that have been widely used for capturing a variety of chemical compounds, including hydrofluorocarbons. For example, Yoshida et al. (JP 2011194337 A) describe a method for removing hydrofluorocarbons such as CH₃F and/or CHF₃ from the exhaust gas discharged from the manufacturing process of a semiconductor or a liquid crystal using a binder-less X type zeolite. Corbin et al. (U.S. Pat. No. 5,523,499) describe a process for purifying a hexafluoroethane product containing CClF₃ and/or CHF₃ impurities using zeolites. Additionally, Thomas et al. (U.S. Pat. No. 7,597,744) describe the use of molecular sieves to reduce the amount of trifluoromethane present in a mixture of trifluoromethane and trifluoroiodomethane. However, zeolites have not been used to capture trifluoromethane produced in the chlorodifluoromethane manufacturing process.

### SUMMARY

In one embodiment, there is provided herein, a method for capturing trifluoromethane from a gaseous mixture comprising the step of: contacting the gaseous mixture with at least one molecular sieve at a pressure of about 0.1 MPa to about 4.8 MPa and a temperature of about 273 K to about 323 K for a period of time sufficient for the molecular sieve to remove at least a portion of the trifluoromethane;

wherein:
(a) the gaseous mixture is a vent stream from a chlorodifluoromethane manufacturing process, said gaseous mixture consisting essentially of trifluoromethane and nitrogen, oxygen, argon, and/or carbon dioxide; and
(b) the molecular sieve has a pore opening of at least about 5 Angstroms and has a Sanderson electronegativity of less than or equal to about 2.75.

### DETAILED DESCRIPTION

As used above and throughout the description of the invention, the following terms, unless otherwise indicated, shall be defined as follows:

The term “gaseous mixture”, as used herein, refers to a mixture of gases in a vent stream from a chlorodifluoromethane manufacturing process. The gaseous mixture consists essentially of trifluoromethane and nitrogen, oxygen, argon, and/or carbon dioxide. The gaseous mixture may also contain small amounts of chlorodifluoromethane and/or HCl, typically less than 5 wt %.

The terms “capture” and “capturing”, as used herein, refer to the removal of at least a portion of the trifluoromethane from a gaseous mixture by sorption by a molecular sieve, such as a zeolite.

Disclosed herein is a method for capturing trifluoromethane from a gaseous mixture in a vent stream from a chlorodifluoromethane manufacturing process using molecular sieves, such as zeolites or activated carbon. The method is useful for reducing emissions of trifluoromethane, which has a high global warming potential (i.e., GWP=11,700 relative to CO₂ GWP=1).

### Molecular Sieves

Molecular sieves are well known in the art and are defined by R. Szosak [Molecular Sieves Principles of Synthesis and Identification, Van Nostrand Reinhold, NY (1989), page 2]. Zeolites, a class of molecular sieves, are crystalline, highly porous materials. They can be generically described as complex aluminosilicates characterized by a three-dimensional pore system. The zeolite framework structure has corner-linked tetrahedra with Al or Si atoms at centers of the tetrahedra and oxygen atoms at the corners. Such tetrahedra are combined in a well-defined repeating structure comprising various combinations of 4-, 6-, 8-, 10-, and 12-membered rings. The resulting framework structure is one of regular channels and cages, which has a pore network that is useful for separation or purification purposes. The size of pore opening is critical to the performance of zeolite in separation or purification applications, since this characteristic determines whether molecules of certain size can enter and exit the zeolite pore system.

The size of the pore opening that controls access to the interior of the zeolites is determined not only by the geometric dimensions of the tetrahedra forming the pore opening, but also by the presence or absence of ions in or near the pore. For example, in the case of zeolite A, access can be restricted by monovalent ions, such as Na⁺ or K⁺, which are situated in or near 8-member ring openings as well as 6-member ring openings. Access can be enhanced by divalent ions, such as Ca²⁺, which are situated only in or near 6-member ring openings. Thus, the potassium and sodium salts of zeolite A exhibit pore openings of about 3 Angstroms and about 4 Angstroms respectively, whereas the calcium salt of zeolite A has a pore opening of about 5 Angstroms.

The Sanderson electronegativity model (see R. T. Sanderson, “Chemical Bonds and Bond Energy”, 2^{nd} ed., Academic Press, New York, 1976; R. T. Sanderson, “Electronegativity and Bond Energy”, J. Amer. Chem. Soc. 1983, 105, 2259-2261; W. J. Mortier, “Zeolite Electronegativity Related to Physicochemical Properties”, J. Catal. 1978, 83, 138-145) furnishes a useful method for classifying inorganic molecular sieves based on their chemical composition. In accordance with this invention the preferential sorption of trifluoromethane by molecular sieves can be correlated with their intermediate electronegativity (i.e., their Sᵢₙₜ, the geometric mean of the electronegativities) as determined by the Sanderson method based upon chemical composition. According to Barthomeuf (D. Barthomeuf, “Acidity and Basicity in Zeolites”, In Catalysis and Adsorption in Zeolites, G. Ohlmann et al., eds., Elsevier (1991), pages 157-169), an apparent Sᵢₙₜ break point between acidity and basicity is at about 3.5 (based on Sanderson (1976)) or 2.6 (based on Sanderson (1983)). In other words, generally, zeolites with Sᵢₙₜ less than about 2.6 (based on Sanderson (1983) tend to exhibit base properties, while those with Sᵢₙₜ greater than about 2.6 are acidic. Example Sᵢₙₜ values are provided in Table 1.

TABLE 1
Intermediate Sanderson Electronegativities
for Selected Molecular Sieves

| | Approximate | |
|---|---|---|
| Molecular Sieve | Composition | Sᵢₙₜ |
| Zeolite 5A (Ca²⁺) | Ca₄Na₄Al₁₂Si₁₂O₄₈ | 2.56 |
| Zeolite A (Sr²⁺) | Sr₄Na₄Al₁₂Si₁₂O₄₈ | 2.52 |
| Zeolite A (Ba²⁺) | Ba₄Na₄Al₁₂Si₁₂O₄₈ | 2.51 |
| Zeolite A (Zn²⁺) | Zn₄Na₄Al₁₂Si₁₂O₄₈ | 2.67 |
| Zeolite A (Cd²⁺) | Cd₄Na₄Al₁₂Si₁₂O₄₈ | 2.66 |
| Zeolite LSX | Na₇₃K₂₂Al₉₅Si₉₇O₃₈₄ | 2.31 |
| Zeolite 13X | Na₈₆Al₈₆Si₁₀₆O₃₈₄ | 2.38 |
| Zeolite NaY | Na₅₆Al₅₆Si₁₃₆O₃₈₄ | 2.58 |
| Zedolite HY | H₅₆Al₅₆Si₁₃₆O₃₈₄ | 2.95 |

Molecular sieves suitable for use in the method disclosed herein have a pore opening of at least about 5 Angstroms and a Sanderson electronegativity of less than or equal to about 2.75 (based on Sanderson (1983)).

In some embodiments, the molecular sieve has a pore opening of about 5 Angstroms to about 9 Angstroms and a Sanderson electronegativity of less than or equal to about 2.75 (based on Sanderson (1983)).

In one embodiment, the molecular sieve is activated carbon.

In some embodiments, the molecular sieve is a zeolite.

In some embodiments, the zeolite is selected from one or more members of the group consisting of zeolite X, zeolite Y, zeolite LSX, and the divalent cation forms of zeolite A, such as Ca²⁺, Sr²⁺, Ba²⁺, Cd²⁺, and Zn²⁺.

In other embodiments, the zeolite is selected from one or more members of the group consisting of zeolite 5A and zeolite 13X.

In some embodiments, the zeolite is zeolite LSX.

Mixtures of any of the aforementioned zeolites may also be used in the method disclosed herein.

Zeolites are typically pre-treated before use by heating, optionally in a dry gas stream. The pre-treatment temperature is typically in the range of from about 100° C. to about 500° C. The dry gas stream is typically dry air or dry nitrogen.

### Method for Capturing Trifluoromethane

The method disclosed herein is useful for capturing trifluoromethane from a gaseous mixture in a vent stream from a chlorodifluoromethane manufacturing process. Chlorodifluoromethane is prepared by reacting chloroform with HF according to the following reaction:

HCCl₃+2HF→HCF₂Cl+2HCl

Trifluoromethane is a by-product of this reaction, typically present at a level of less than 5 wt %. The chlorodifluoromethane is separated from the trifluoromethane by a distillation process, resulting in a mixture containing primarily trifluoromethane and HCl. The HCl is removed from the mixture by a scrubbing process which utilizes water. Residual trifluoromethane dissolved in the scrubbing solution is removed using inert gas such as air, argon, or nitrogen, resulting in a gaseous mixture consisting essentially of trifluoromethane and nitrogen, oxygen, argon, and/or carbon dioxide. The gaseous mixture may also contain small amounts of chlorodifluoromethane and/or HCl, typically less than 5 wt %. This gaseous mixture is typically vented into the atmosphere as a vent stream. However, it is desirable to capture the trifluoromethane in the vent stream to prevent its release into the atmosphere because of the very high global warming potential of trifluoromethane (i.e., GWP=11,700 relative to CO₂ GWP=1).

In the method disclosed herein, the gaseous mixture in the vent stream from a chlorodifluoromethane manufacturing process is contacted with at least one molecular sieve, described above, at a pressure of about 0.1 MPa to about 4.8 MPa, and a temperature of about 273 K to about 323 K for a period of time sufficient for the molecular sieve to remove at least a portion of the trifluoromethane present in the gaseous mixture. Ideally, substantially all of the trifluoromethane is removed by the molecular sieve. Suitable conditions for the capture of the trifluoromethane from the gaseous mixture may be determined by one skilled in the art using routine experimentation. In some embodiments, the gaseous mixture is contacted with the molecular sieve at a pressure of about 0.5 MPa to about 4.5 MPa, more particularly about 1.0 MPa to about 4.5 MPa, and more particularly about 2.0 MPa to about 4.5 MPa.

In some embodiments, the gaseous mixture is contacted with the molecular sieve at a temperature of about 283 K to about 323 K, more particularly about 298 K to about 323 K.

In the method disclosed herein, the molecular sieve may be contained in a stationary packed bed through which the gaseous mixture from the vent stream is passed. Alternatively, the molecular sieve may be used in the form of a countercurrent moving bed or in a fluidized bed for contacting the gaseous mixture from the vent stream. In these embodiments, the trifluoromethane is captured by the molecular sieve in the bed and the remaining components of the gaseous mixture pass through.

After capture of the trifluoromethane, the molecular sieve may be regenerated by heating with steam to release the sorbed trifluoromethane and reused in the method. The released trifluoromethane may be incinerated or liquefied by pressurizing for storage. Alternatively, the molecular sieve with the sorbed trifluoromethane may be incinerated for disposal.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “h” means hour(s), “mL” means milliliter(s), “μL” means microliter(s), “g” means gram(s), “mg” means milligram(s), “μg” means microgram(s), “Pa” means pascal(s), “kPa” means kilopascal(s), and “MPa” means megapascal(s).

### Materials

Trifluoromethane (R-23, CHF₃, purity >99.995%, molecular weight 70.014 g mol⁻¹, CAS no. 75-46-7) was purchased from GTS-Welco (Allentown, Pa.). Zeolite 5A (theoretical “pseudo” unit cell composition Ca₄Na₄[(AlO₂)₁₂(SiO₂)₁₂].xH₂O, molecular weight 1681.05, CAS no. 69912-79-4) and Zeolite 13X ((theoretical unit cell composition Na₈₆[(AlO₂)₈₆(SiO₂)₁₀₆].xH₂O, molecular weight 13418.38 g mol⁻¹, CAS no. 63231-69-6) were purchased from Aldrich (Milwaukee, Wis.). Zeolite LSX ((theoretical unit cell composition Na₇₃K₂₂(AlO₂)₉₅(SiO₂)₉₇.xH₂O, molecular weight 13969.72 g mol⁻¹, CAS no. 68989-22-0) was obtained from Zeochem, L.L.C. (Louisville, Ky.).

The zeolites were activated by heating a 2 gram sample under vacuum at 648 K for 12 h. The heating rate to reach this temperature was 30 K min⁻¹.

### Example 1

### Sorption of Trifluoromethane by Zeolite 5A

This Example illustrates the sorption of trifluoromethane by Zeolite 5A at temperatures of 298 K and 323 K. The sorption was measured using a gravimetric microbalance.

The sorption measurements were made using a gravimetric microbalance (IGA-003 Multicomponent Analyzer, Hiden Isochema Ltd., Warrington WA5 7TN UK). The IGA design integrates precise computer-control and measurement of weight change, pressure and temperature to enable fully automatic and reproducible determination of gas sorption isotherms and isobars. The microbalance consists of an electrobalance with sample and counterweight components inside a stainless steel pressure-vessel. The balance has a weigh range of 0-100 mg with a resolution of 0.1 μg.

Approximately 50 mg of the zeolite was loaded into a quartz glass container inside the microbalance. The reactor was sealed and evacuated. The zeolites were further dried by heating for 24 h at 323 K until no noticeable mass change was detected.

An enhanced pressure stainless steel (SS316LN) reactor capable of operation to 2.0 MPa and 773.15 K was installed. The advantages of using a microbalance include the minimal sample size (<100 mg) required, the ability to automate the measurement process to take several PTx data, and the flexibility to measure both sorption and desorption isotherms. When done properly, the gravimetric analysis provides a direct an accurate method for assessing both gas solubility and diffusivity. Two critical factors that must be considered include properly correcting for the buoyancy effects of the system and allowing sufficient time to reach equilibrium (i.e., no mixing is possible).

The IGA-003 can operate in both dynamic and static modes. All sorption measurements were performed in static mode. Static mode operation introduces gas into the top of the balance away from the sample, and both the admittance and exhaust valves control the set-point pressure. The sample temperature was measured with a resistance temperature device (RTD) with an accuracy of ±0.1 K. The RTD was calibrated using a standard platinum resistance thermometer (SPRT model 5699, Hart Scientific, American Fork, Utah, range 73 to 933 K) and readout (Blackstack model 1560 with SPRT module 2560). The Blackstack instrument and SPRT are a certified secondary temperature standard with a NIST traceable accuracy to ±0.005 K. Two isotherms of about 298 and 323 K were measured beginning with 298 K. Two pressure sensors were used for the measurements. Pressures from 10⁻⁴ to 10⁻² MPa were measured using a capacitance manometer (MKS, model Baratron 626A) with an accuracy of ±0.015 kPa. Pressures from 10⁻² to 2.0 MPa were measured using a piezo-resistive strain gauge (Druck, model PDCR4010) with an accuracy of ±0.8 kPa. The Druck low-pressure transducer was calibrated against a Paroscientific Model 765-15A (Redmond, Wash.) pressure transducer (range 0 to 0.102 MPa, part no. 1100-001, serial no. 104647). The Druck high-pressure transducer was calibrated against a Paroscientific Model 765-1K (Redmond, Wash.) pressure transducer (range 0 to 6.805 MPa, part no. 1100-017, serial no. 101314). These instruments are also a NIST certified secondary pressure standard with a traceable accuracy of 0.008% of full scale. The upper pressure limit of the microbalance reactor was 2.0 MPa, and several isobars up to 2.0 MPa (0.01, 0.02, 0.03, 0.04, 0.05, 0.06, 0.07, 0.08, 0.09, 0.1, 0.25, 0.50, 0.75, 1.0, 1.25, 1.5, 1.75 and 2.0 MPa) were measured. To ensure sufficient time to reach equilibrium, a minimum time of 10 h and a maximum time of 20 h were set for isotherms measured at 298 and 323 K. The total uncertainties in the solubility data due to both random and systematic errors have been estimated to be less than 0.006 mole fraction at given T and P. The equivalent uncertainty in molality for Zeolite 5A was 0.0036 mol·kg⁻¹ at given T and P.

The corrected sorption (PTx) data for trifluoromethane by Zeolite 5A is shown in Table 2. In the table, x₁ is the mole fraction of trifluoromethane. Desorption isotherms were also measured at 298 and 323 K and the (PTx) data are included in Table 2. The trifluoromethane mass uptake versus time for sorption and desorption experiments between 0 and 2.0 MPa at 298 and 323 K indicate the sorption is reversible for Zeolite 5A.

TABLE 2
Sorption Data for Trifluoromethane by Zeolite 5A

| | | | | |
|---|---|---|---|---|
| | | | | Molality/mol |
| T/K | P/MPa | wt % | 100 x₁ | kg⁻¹ |

Sorption

| | | | | |
|---|---|---|---|---|
| 298.1 | 0.0024 | 6.4 | 62.1 | 0.98 |
| 298.1 | 0.0053 | 9.6 | 71.8 | 1.52 |
| 298.1 | 0.0069 | 10.3 | 73.3 | 1.63 |
| 298.1 | 0.0109 | 12.5 | 77.4 | 2.03 |
| 298.1 | 0.0202 | 14.4 | 80.1 | 2.40 |
| 298.1 | 0.0301 | 15.6 | 81.6 | 2.64 |
| 298.1 | 0.0400 | 16.1 | 82.2 | 2.75 |
| 298.1 | 0.0503 | 16.9 | 83.0 | 2.91 |
| 298.1 | 0.0750 | 17.4 | 83.5 | 3.01 |
| 298.1 | 0.1002 | 18.1 | 84.2 | 3.17 |
| 298.1 | 0.2501 | 19.8 | 85.5 | 3.52 |
| 298.1 | 0.4999 | 21.2 | 86.6 | 3.84 |
| 298.1 | 0.7499 | 22.3 | 87.3 | 4.11 |
| 298.1 | 1.0026 | 23.3 | 88.0 | 4.35 |
| 298.1 | 1.2513 | 24.3 | 88.5 | 4.59 |
| 298.1 | 1.5007 | 25.3 | 89.0 | 4.84 |
| 298.1 | 1.7507 | 26.3 | 89.5 | 5.09 |
| 298.1 | 2.0012 | 27.3 | 90.0 | 5.35 |

Sorption

| | | | | |
|---|---|---|---|---|
| 323.1 | 0.0026 | 5.8 | 59.8 | 0.88 |
| 323.1 | 0.0057 | 9.6 | 71.9 | 1.52 |
| 323.1 | 0.0090 | 10.9 | 74.7 | 1.76 |
| 323.1 | 0.0113 | 12.1 | 76.7 | 1.96 |
| 323.1 | 0.0209 | 13.6 | 79.1 | 2.25 |
| 323.1 | 0.0307 | 14.6 | 80.4 | 2.44 |
| 323.1 | 0.0402 | 15.1 | 81.1 | 2.55 |
| 323.1 | 0.0503 | 15.6 | 81.6 | 2.64 |
| 323.1 | 0.0752 | 16.3 | 82.4 | 2.78 |
| 323.1 | 0.1000 | 16.9 | 83.0 | 2.90 |
| 323.1 | 0.2498 | 18.7 | 84.7 | 3.29 |
| 323.1 | 0.4997 | 20.2 | 85.9 | 3.62 |
| 323.2 | 0.7495 | 21.4 | 86.7 | 3.88 |
| 323.1 | 0.9994 | 22.3 | 87.3 | 4.11 |
| 323.1 | 1.2520 | 23.3 | 87.9 | 4.33 |
| 323.1 | 1.4992 | 24.1 | 88.4 | 4.54 |
| 323.1 | 1.7524 | 25.0 | 88.9 | 4.75 |
| 323.2 | 2.0030 | 25.8 | 89.3 | 4.96 |

Desorption

| | | | | |
|---|---|---|---|---|
| 323.1 | 0.9991 | 22.3 | 87.4 | 4.11 |
| 323.1 | 0.0998 | 17.1 | 83.2 | 2.94 |

### Example 2

### Sorption of Trifluoromethane by Zeolite 13X

This Example illustrates the sorption of trifluoromethane on Zeolite 13X at temperatures of 298 K and 323 K. The sorption was measured using a gravimetric microbalance using the method described in Example 1.

The equivalent uncertainties in molality for Zeolite 13X was 0.0004 mol·kg⁻¹ at given T and P. The corrected sorption (PTx) data for trifluoromethane by Zeolite 13X is shown in Table 3.

TABLE 3
Sorption Data for Trifluoromethane by Zeolite 13X

| | | | | |
|---|---|---|---|---|
| | | | | Molality/mol |
| T/K | P/MPa | wt % | 100 x₁ | kg⁻¹ |

Sorption

| | | | | |
|---|---|---|---|---|
| 298.1 | 0.0010 | 12.3 | 96.4 | 2.01 |
| 298.1 | 0.0020 | 14.3 | 97.0 | 2.39 |
| 298.1 | 0.0030 | 15.4 | 97.2 | 2.60 |
| 298.1 | 0.0040 | 16.0 | 97.4 | 2.73 |
| 298.1 | 0.0050 | 16.5 | 97.4 | 2.83 |
| 298.1 | 0.0060 | 16.9 | 97.5 | 2.91 |
| 298.1 | 0.0070 | 17.2 | 97.6 | 2.97 |
| 298.3 | 0.0077 | 17.4 | 97.6 | 3.00 |
| 298.1 | 0.0080 | 17.5 | 97.6 | 3.03 |
| 298.1 | 0.0090 | 17.7 | 97.6 | 3.07 |
| 298.1 | 0.0111 | 18.0 | 97.7 | 3.15 |
| 298.1 | 0.0301 | 19.3 | 97.9 | 3.41 |
| 298.1 | 0.0400 | 19.7 | 97.9 | 3.50 |
| 298.1 | 0.0501 | 20.0 | 98.0 | 3.57 |
| 298.1 | 0.1004 | 20.9 | 98.1 | 3.78 |
| 298.1 | 0.2517 | 22.3 | 98.2 | 4.10 |
| 298.1 | 0.5010 | 23.6 | 98.3 | 4.41 |
| 298.1 | 0.7505 | 24.7 | 98.4 | 4.68 |
| 298.1 | 1.0005 | 25.6 | 98.5 | 4.92 |
| 298.1 | 1.2508 | 26.6 | 98.6 | 5.16 |
| 298.1 | 1.5015 | 27.5 | 98.6 | 5.42 |
| 298.1 | 1.7507 | 28.5 | 98.7 | 5.68 |
| 298.1 | 2.0009 | 29.4 | 98.8 | 5.96 |

Sorption

| | | | | |
|---|---|---|---|---|
| 323.1 | 0.0005 | 5.8 | 92.2 | 0.88 |
| 323.1 | 0.0010 | 7.9 | 94.2 | 1.22 |
| 323.1 | 0.0020 | 10.2 | 95.6 | 1.62 |
| 323.1 | 0.0030 | 11.5 | 96.1 | 1.85 |
| 323.1 | 0.0040 | 12.5 | 96.5 | 2.04 |
| 323.1 | 0.0050 | 13.3 | 96.7 | 2.18 |
| 323.1 | 0.0062 | 13.9 | 96.9 | 2.30 |
| 323.1 | 0.0075 | 14.4 | 97.0 | 2.41 |
| 323.2 | 0.0200 | 16.6 | 97.5 | 2.85 |
| 323.1 | 0.0301 | 17.5 | 97.6 | 3.02 |
| 323.1 | 0.0400 | 18.0 | 97.7 | 3.13 |
| 323.1 | 0.0503 | 18.4 | 97.7 | 3.22 |
| 323.1 | 0.1001 | 18.9 | 97.8 | 3.32 |
| 323.2 | 0.2501 | 20.5 | 98.0 | 3.69 |
| 323.1 | 0.5010 | 22.0 | 98.2 | 4.03 |
| 323.1 | 0.7518 | 23.1 | 98.3 | 4.29 |
| 323.1 | 1.0014 | 24.1 | 98.4 | 4.53 |
| 323.1 | 1.2508 | 25.0 | 98.5 | 4.75 |
| 323.1 | 1.5009 | 25.9 | 98.5 | 4.99 |
| 323.1 | 1.7505 | 26.8 | 98.6 | 5.22 |
| 323.2 | 2.0003 | 27.6 | 98.7 | 5.44 |

### Example 3

### Sorption of Trifluoromethane by Zeolite LSX

This Example illustrates the sorption of trifluoromethane by Zeolite LSX at temperatures of 298 K and 323 K. The sorption was measured using a gravimetric microbalance using the method described in Example 1.

The equivalent uncertainties in molality for Zeolite LSX was 0.0004 mol·kg⁻¹ at given T and P. The corrected sorption (PTx) data for trifluoromethane by Zeolite LSX is shown in Table 4. Desorption isotherms were also measured at 298 and 323 K and the (PTx) data are included in Table 4. The trifluoromethane mass uptake versus time for sorption and desorption experiments between 0 and 2.0 MPa at 298 and 323 K indicate the sorption is reversible for Zeolite LSX.

TABLE 4
Sorption Data for Trifluoromethane on Zeolite LSX

| | | | | |
|---|---|---|---|---|
| | | | | Molality/mol |
| T/K | P/MPa | wt % | 100 x₁ | kg⁻¹ |

Sorption

| | | | | |
|---|---|---|---|---|
| 298.1 | 0.0010 | 17.2 | 97.6 | 2.96 |
| 298.1 | 0.0020 | 18.0 | 97.7 | 3.13 |
| 298.1 | 0.0030 | 18.4 | 97.8 | 3.22 |
| 298.1 | 0.0040 | 18.7 | 97.8 | 3.28 |
| 298.1 | 0.0050 | 18.9 | 97.8 | 3.33 |
| 298.1 | 0.0051 | 18.9 | 97.8 | 3.34 |
| 298.1 | 0.0060 | 19.1 | 97.9 | 3.37 |
| 298.1 | 0.0070 | 19.2 | 97.9 | 3.40 |
| 298.1 | 0.0080 | 19.4 | 97.9 | 3.43 |
| 297.8 | 0.0090 | 19.5 | 97.9 | 3.47 |
| 298.1 | 0.0106 | 19.6 | 97.9 | 3.48 |
| 298.1 | 0.0103 | 19.7 | 97.9 | 3.50 |
| 298.1 | 0.0205 | 20.2 | 98.0 | 3.62 |
| 298.1 | 0.0248 | 20.5 | 98.0 | 3.68 |
| 298.1 | 0.0303 | 20.6 | 98.1 | 3.71 |
| 298.1 | 0.0402 | 20.9 | 98.1 | 3.78 |
| 298.1 | 0.0501 | 21.2 | 98.1 | 3.84 |
| 298.1 | 0.0498 | 21.3 | 98.1 | 3.86 |
| 298.1 | 0.0999 | 22.0 | 98.2 | 4.03 |
| 298.1 | 0.0998 | 22.1 | 98.2 | 4.05 |
| 298.1 | 0.2500 | 23.3 | 98.3 | 4.34 |
| 298.1 | 0.4999 | 24.6 | 98.4 | 4.65 |
| 298.1 | 0.7494 | 25.6 | 98.5 | 4.91 |
| 298.1 | 1.0001 | 26.5 | 98.6 | 5.16 |
| 298.1 | 1.2498 | 27.4 | 98.7 | 5.40 |
| 298.1 | 1.5002 | 28.3 | 98.7 | 5.64 |
| 298.1 | 1.7496 | 29.2 | 98.8 | 5.90 |
| 298.1 | 1.9996 | 30.2 | 98.8 | 6.17 |

Desorption

| | | | | |
|---|---|---|---|---|
| 298.1 | 0.0998 | 22.1 | 98.2 | 4.05 |
| 298.1 | 0.0498 | 21.3 | 98.1 | 3.86 |

Sorption

| | | | | |
|---|---|---|---|---|
| 323.1 | 0.0010 | 15.4 | 97.3 | 2.61 |
| 323.1 | 0.0020 | 16.6 | 97.5 | 2.84 |
| 323.1 | 0.0030 | 17.2 | 97.6 | 2.97 |
| 323.2 | 0.0040 | 17.6 | 97.7 | 3.05 |
| 323.2 | 0.0050 | 17.9 | 97.7 | 3.11 |
| 323.1 | 0.0056 | 18.1 | 97.7 | 3.15 |
| 323.1 | 0.0060 | 18.1 | 97.7 | 3.16 |
| 323.2 | 0.0070 | 18.3 | 97.8 | 3.20 |
| 323.1 | 0.0080 | 18.5 | 97.8 | 3.24 |
| 323.1 | 0.0090 | 18.6 | 97.8 | 3.27 |
| 323.1 | 0.0102 | 18.7 | 97.8 | 3.29 |
| 323.1 | 0.0099 | 18.8 | 97.8 | 3.30 |
| 323.1 | 0.0106 | 18.8 | 97.8 | 3.30 |
| 323.1 | 0.0204 | 19.5 | 97.9 | 3.45 |
| 323.1 | 0.0248 | 19.7 | 97.9 | 3.50 |
| 323.1 | 0.0304 | 19.9 | 98.0 | 3.55 |
| 323.1 | 0.0402 | 20.2 | 98.0 | 3.62 |
| 323.1 | 0.0501 | 20.4 | 98.0 | 3.67 |
| 323.1 | 0.0498 | 20.4 | 98.0 | 3.67 |
| 323.1 | 0.0998 | 21.2 | 98.1 | 3.85 |
| 323.1 | 0.1000 | 21.3 | 98.1 | 3.85 |
| 323.1 | 0.2499 | 22.6 | 98.3 | 4.16 |
| 323.1 | 0.5000 | 23.9 | 98.4 | 4.48 |
| 323.1 | 0.7499 | 24.9 | 98.5 | 4.73 |
| 323.2 | 0.9996 | 25.8 | 98.5 | 4.96 |
| 323.1 | 1.2515 | 26.6 | 98.6 | 5.18 |
| 323.2 | 1.5003 | 27.4 | 98.7 | 5.39 |
| 323.2 | 1.7526 | 28.2 | 98.7 | 5.61 |
| 323.1 | 2.0026 | 29.0 | 98.8 | 5.84 |

Desorption

| | | | | |
|---|---|---|---|---|
| 323.1 | 0.0998 | 21.2 | 98.1 | 3.85 |
| 323.1 | 0.0498 | 20.5 | 98.0 | 3.67 |
| 323.1 | 0.0049 | 17.9 | 97.7 | 3.12 |
| 323.2 | 0.0010 | 15.5 | 97.3 | 2.61 |

### Example 4

### Sorption of Trifluoromethane in Activated Carbon

This Example illustrates the sorption of trifluoromethane by activated carbon at temperatures of 298 K and 323 K. The sorption was measured using a gravimetric microbalance using the method described in Example 1.

The activated carbon was synthesized from coal tar pitch. The pitch was stabilized by heating to 573 K. The carbon was activated by heating to about 1153 to 1173 K in the presence of potassium hydroxide (KOH) vapors. In order to dry the carbon and remove any residual KOH vapor or adsorbed gases from the pores, the activated carbon was heated in a vertical tube furnace at 623 K for 24 hours under vacuum.

The surface area and pore volume were measured by nitrogen adsorption/desorption measurements, performed at 77 K on a Micromeritics ASAP model 2420 porosimeter. Samples were degased at 423 K overnight prior to data collection. Surface area measurements utilized a five-point adsorption isotherm collected over 0.05 to 0.20 P/P₀ (P₀=nitrogen saturation pressure) and analyzed via the BET method. (Brunauer et al., J. Amer. Chem. Soc. 60, 309-319, 1938) Total pore volume was determined by a single point measurement at P/P₀=0.995. The BET specific surface area was 3206 m² g⁻¹ with a Type I isotherm. The BET model has inaccuracies for micropore systems which can lead to condensation even at low relative pressure and, correspondingly, to an overestimation of the surface area (Kaneko, et al., Carbon 30, 1075-1088 1992). The BET specific surface area is a reproducible measurement, characteristic of the material, but possibly an overestimation of the total surface area. The total pore volume is 1.68 cm³ g⁻¹ with an average pore diameter of 2.0 nm.

The equivalent uncertainty in molality for activated carbon was 0.5 mol kg⁻¹ at given T and P. The corrected solubility (PTx) data for R-23 in the activated carbon is shown in Table 5. Desorption isotherms were also measured at 298 and 323 K and the (PTx) data are included in Table 5. The R-23 mass uptake between 0 and 2.0 MPa at 298 and 323 K indicate the sorption is reversible.

TABLE 5
Sorption Data for Trifluoromethane on Activated Carbon

| | | | | |
|---|---|---|---|---|
| | | | | Molality/mol |
| T/K | P/MPa | wt % | 100 x₁ | kg⁻¹ |

Sorption

| | | | | |
|---|---|---|---|---|
| 298.1 | 0.0010 | 0.76 | 0.0013 | 0.109 |
| 298.1 | 0.0020 | 1.39 | 0.0024 | 0.199 |
| 298.1 | 0.0030 | 1.96 | 0.0034 | 0.280 |
| 298.1 | 0.0040 | 2.45 | 0.0043 | 0.350 |
| 298.1 | 0.0050 | 2.93 | 0.0051 | 0.418 |
| 298.1 | 0.0060 | 3.36 | 0.0059 | 0.480 |
| 298.1 | 0.0070 | 3.76 | 0.0067 | 0.537 |
| 298.1 | 0.0080 | 4.16 | 0.0074 | 0.594 |
| 298.1 | 0.0090 | 4.55 | 0.0081 | 0.650 |
| 298.1 | 0.0102 | 4.88 | 0.0087 | 0.697 |
| 298.1 | 0.0248 | 9.22 | 0.0171 | 1.317 |
| 298.1 | 0.0498 | 14.54 | 0.0283 | 2.077 |
| 298.1 | 0.0996 | 21.90 | 0.0459 | 3.128 |
| 298.1 | 0.2438 | 34.06 | 0.0814 | 4.865 |
| 298.1 | 0.4531 | 42.87 | 0.1140 | 6.123 |
| 298.1 | 0.7492 | 49.54 | 0.1442 | 7.076 |
| 298.1 | 0.9972 | 53.10 | 0.1626 | 7.584 |
| 298.1 | 1.2489 | 55.86 | 0.1784 | 7.978 |
| 298.1 | 1.4992 | 58.06 | 0.1919 | 8.293 |
| 298.1 | 1.7494 | 59.90 | 0.2039 | 8.555 |
| 298.1 | 1.9989 | 61.45 | 0.2147 | 8.777 |

Desorption

| | | | | |
|---|---|---|---|---|
| 298.1 | 1.9963 | 61.47 | 0.2148 | 8.780 |
| 298.1 | 1.7476 | 59.91 | 0.2040 | 8.557 |
| 298.2 | 0.4431 | 42.60 | 0.1129 | 6.084 |
| 298.1 | 0.2499 | 34.49 | 0.0828 | 4.926 |
| 298.1 | 0.0998 | 21.98 | 0.0461 | 3.139 |
| 298.1 | 0.0748 | 18.64 | 0.0378 | 2.662 |
| 298.1 | 0.0499 | 14.57 | 0.0284 | 2.081 |
| 298.1 | 0.0249 | 9.30 | 0.0173 | 1.328 |
| 298.1 | 0.0099 | 4.86 | 0.0087 | 0.694 |
| 298.1 | 0.0093 | 4.72 | 0.0084 | 0.674 |
| 298.1 | 0.0080 | 4.24 | 0.0075 | 0.606 |
| 298.1 | 0.0070 | 3.85 | 0.0068 | 0.550 |
| 298.1 | 0.0060 | 3.45 | 0.0061 | 0.493 |
| 298.1 | 0.0050 | 3.01 | 0.0053 | 0.430 |
| 298.1 | 0.0040 | 2.53 | 0.0044 | 0.361 |
| 298.1 | 0.0030 | 2.03 | 0.0035 | 0.290 |
| 298.1 | 0.0020 | 1.47 | 0.0026 | 0.210 |
| 298.1 | 0.0010 | 0.84 | 0.0015 | 0.120 |

Sorption

| | | | | |
|---|---|---|---|---|
| 323.2 | 0.0010 | 0.23 | 0.0004 | 0.033 |
| 323.1 | 0.0020 | 0.55 | 0.0010 | 0.079 |
| 323.1 | 0.0030 | 0.85 | 0.0015 | 0.121 |
| 323.2 | 0.0040 | 1.14 | 0.0020 | 0.163 |
| 323.2 | 0.0050 | 1.40 | 0.0024 | 0.200 |
| 323.1 | 0.0060 | 1.65 | 0.0029 | 0.236 |
| 323.2 | 0.0070 | 1.90 | 0.0033 | 0.271 |
| 323.1 | 0.0080 | 2.15 | 0.0038 | 0.307 |
| 323.1 | 0.0090 | 2.38 | 0.0042 | 0.340 |
| 323.2 | 0.0099 | 2.53 | 0.0044 | 0.361 |
| 323.2 | 0.0248 | 5.37 | 0.0096 | 0.767 |
| 323.2 | 0.0498 | 9.05 | 0.0168 | 1.293 |
| 323.1 | 0.0746 | 12.00 | 0.0229 | 1.714 |
| 323.2 | 0.0999 | 14.63 | 0.0286 | 2.090 |
| 323.1 | 0.2488 | 25.37 | 0.0551 | 3.624 |
| 323.1 | 0.4977 | 35.54 | 0.0864 | 5.076 |
| 323.2 | 0.7493 | 41.63 | 0.1090 | 5.946 |
| 323.1 | 0.9995 | 45.77 | 0.1265 | 6.537 |
| 323.3 | 1.2428 | 48.80 | 0.1405 | 6.970 |
| 323.1 | 1.4988 | 51.36 | 0.1534 | 7.336 |
| 323.1 | 1.7492 | 53.41 | 0.1643 | 7.628 |
| 323.0 | 1.9927 | 55.13 | 0.1741 | 7.874 |

Desorption

| | | | | |
|---|---|---|---|---|
| 323.2 | 1.9912 | 55.13 | 0.1741 | 7.874 |
| 323.2 | 1.7384 | 53.37 | 0.1641 | 7.623 |
| 323.2 | 1.4931 | 51.34 | 0.1532 | 7.333 |
| 323.1 | 1.2496 | 48.88 | 0.1409 | 6.981 |
| 323.2 | 0.9997 | 45.78 | 0.1265 | 6.539 |
| 323.2 | 0.7457 | 41.57 | 0.1088 | 5.937 |
| 323.1 | 0.4995 | 35.63 | 0.0867 | 5.089 |
| 323.2 | 0.2499 | 25.50 | 0.0555 | 3.642 |
| 323.2 | 0.0999 | 14.66 | 0.0286 | 2.094 |
| 323.1 | 0.0749 | 12.12 | 0.0231 | 1.731 |
| 323.1 | 0.0498 | 9.11 | 0.0169 | 1.301 |
| 323.1 | 0.0249 | 5.45 | 0.0098 | 0.778 |
| 323.1 | 0.0099 | 2.58 | 0.0045 | 0.368 |
| 323.2 | 0.0090 | 2.43 | 0.0042 | 0.347 |
| 323.2 | 0.0080 | 2.21 | 0.0039 | 0.316 |
| 323.2 | 0.0070 | 1.98 | 0.0035 | 0.283 |
| 323.1 | 0.0060 | 1.72 | 0.0030 | 0.246 |
| 323.1 | 0.0050 | 1.48 | 0.0026 | 0.211 |
| 323.2 | 0.0040 | 1.21 | 0.0021 | 0.173 |
| 323.1 | 0.0030 | 0.93 | 0.0016 | 0.133 |
| 323.1 | 0.0020 | 0.63 | 0.0011 | 0.090 |
| 323.2 | 0.0010 | 0.31 | 0.0005 | 0.044 |

What is claimed is:

1. A method for capturing trifluoromethane from a gaseous mixture comprising the step of: contacting the gaseous mixture with at least one molecular sieve at a pressure of about 0.1 MPa to about 4.8 MPa and a temperature of about 273 K to about 323 K for a period of time sufficient for the molecular sieve to remove at least a portion of the trifluoromethane;
wherein:
(a) the gaseous mixture is a vent stream from a chlorodifluoromethane manufacturing process, said gaseous mixture consisting essentially of trifluoromethane and nitrogen, oxygen, argon, and/or carbon dioxide; and
(b) the molecular sieve has a pore opening of at least about 5 Angstroms and has a Sanderson electronegativity of less than or equal to about 2.75.

2. The method of claim 1, wherein the molecular sieve has a pore opening of about 5 Angstroms to about 9 Angstroms.

3. The method of claim 1, wherein the molecular sieve is a zeolite.

4. The method of claim 3, wherein the zeolite is selected from one or more members of the group consisting of zeolite X, zeolite Y, zeolite LSX, and the divalent cation forms of zeolite A.

5. The method of claim 3, wherein the zeolite is selected from one or more members of the group consisting of zeolite 5A and zeolite 13X.

6. The method of claim 3, wherein the zeolite is zeolite LSX.

7. The method of claim 1, wherein the temperature is about 298 K to about 323 K.

8. The method of claim 1, wherein the pressure is about 1.0 MPa to about 4.5 MPa.

9. The method of claim 1, wherein the molecular sieve is activated carbon.
